(19) United States
(12) Patent Application Publication
(10) Pub. No.: US 2024/0277292 A1
(43) Pub. Date: Aug. 22, 2024

(54) OPTICAL SENSOR TAPE

(71) Applicant: Masimo Corporation, Irvine, CA (US)

(72) Inventors: Massi Joe E. Kiani, Laguna Niguel, CA (US); Ammar Al-Ali, San Juan Capistrano, CA (US); Benjamin C. Triman, Rancho Santa Margarita, CA (US); Chad A. DeJong, Los Angeles, CA (US)

(21) Appl. No.: 18/650,832

(22) Filed: Apr. 30, 2024

Related U.S. Application Data
(63) Continuation of application No. 17/518,427, filed on Nov. 3, 2021, now Pat. No. 12,004,877.
Application No. 17/518,427 is a continuation of application No. 15/582,082, filed on Apr. 28, 2017, now Pat. No. 11,191,484.
(60) Provisional application No. 62/329,451, filed on Apr. 29, 2016.

(51) Int. Cl. A61B 5/00; A61B 5/1455

(52) U.S. Cl. CPC A61B 5/6833; A61B 5/14551

(57) ABSTRACT

Various sensor tapes can improve securing of a non-invasive optical sensor to a surface of a medium for taking noninvasive measurement of characteristics of the medium. The sensor tape can taper from a wide end to a narrow end. The sensor tape can transition from a wide portion to a narrow portion in a step-like change or slope. The sensor tape can have staggered portions. The various tapes can be used with an L-shaped sensor. The various tapes can increase contact surface between the surface of the medium and an adhesive side of the tape so as to reduce motion-induced noise.

### INCORPORATION BY REFERENCE TO ANY PRIORITY APPLICATIONS

The present application is a continuation of U.S. patent application Ser. No. 17/518,427, filed Nov. 3, 2021, and titled “OPTICAL SENSOR TAPE”, which is a continuation of U.S. patent application Ser. No. 15/582,082, filed Apr. 28, 2017, and titled “OPTICAL SENSOR TAPE”, and issued as U.S. Pat. No. 11,191,484, which claims benefit of priority to U.S. Provisional Application No. 62/329,451, filed Apr. 29, 2016. The disclosure of each of these applications is incorporated herein in its entirety for all purposes. Any and all applications for which a foreign or domestic priority claim is identified in the Application Data Sheet as filed with the present application are hereby incorporated by reference under 37 CFR 1.57.

### FIELD OF THE DISCLOSURE

The present disclosure relates to low-noise optical probes which may be used to sense optical energy passed through a medium to determine the characteristics of the medium.

### BACKGROUND

Pulse oximetry—a noninvasive, widely accepted form of oximetry—relies on a sensor attached externally to a patient to output signals indicative of various physiological parameters, such as a patient's constituents or analytes, including, for example, oxygen saturation (SpO2), hemoglobin (Hb), blood pressure (BP), pulse rate (PR), perfusion index (PI), Pleth Variable Index (PVI), carbon monoxide saturation (HbCO), methemoglobin saturation (HbMet), fractional saturations, total hematocrit, billirubins, or the like. As such a pulse oximeter is one of a variety of patient monitors that help provide monitoring of a patient's physiological characteristics.

Pulse oximeters are available from Masimo Corporation (“Masimo”) of Irvine, California. Moreover, some exemplary portable and other oximeters are disclosed in at least U.S. Pat. Nos. 6,770,028, 6,658,276, 6,157,850, 6,002,952, and 5,769,785, which are owned by Masimo, and are incorporated by reference herein. Such oximeters have gained rapid acceptance in a wide variety of medical applications, including surgical wards, intensive care and neonatal units, general wards, home care, physical training, and virtually all types of monitoring scenarios.

### SUMMARY

A pulse oximeter sensor generally includes one or more energy emission devices, such as specific wavelength emitting light emitting diodes (“LED”), and one or more energy detection devices. The sensor is generally attached to a measurement site such as a patient's finger, toe, ear, ankle, or the like. An attachment mechanism positions the emitters and detector, collectively called an optical probe, proximal to the measurement site such that the emitters project energy into the tissue, blood vessels, and capillaries of the measurement site, which in turn attenuate the energy. The detector then detects that attenuated energy. The detector communicates at least one signal indicative of the detected attenuated energy to one or more digital signal processors, for calculating, among other things, one or more physiological parameters of the measurement site.

The present disclosure discloses an improved sensor tape for securing a non-invasive optical sensor, such as a pulse oximeter sensor, to a surface of a medium for taking noninvasive measurement of characteristics of the medium. The sensor tapes of the present disclosure can increase contact surface between the surface of the medium and an adhesive side of the tape in order to increase tape adhesion to the medium and to reduce motion-induced noise. The sensor tapes of the present disclosure can be disposable and lost cost. The sensor tapes of the present disclosure can also be manufactured in a manner that maximizes the amount of material used so as to keep material cost low.

One type of disposable sensor uses an “L”-shaped configuration. This type of configuration is generally used for infant patients so that the sensor can be used in a variety of measurement sites on the infant. As the tape is applied to the patient, the tape is often wound around a patient measurement site and later portions of the tape are adhered to the back of previous portions of the tape. Although the present disclosure is described mainly with respect to an L-shaped tape sensor, the embodiments of sensor tapes in this disclosure are not limited to being used with an L-shaped sensor, but are applicable to any type of sensor shapes and configurations.

In some embodiments, a sensor tape for securing a non-invasive optical sensor to a surface of a medium for taking physiological measurements is disclosed. The sensor tape can comprise a first end with a first width, a second end with a second width, the second width greater than the first width, and a flexible tape portion between the first and second ends, the tape portion including an adhesive surface and a non-adhesive surface. The sensor tape can be tapered such that a width of the tape decreases gradually from the second end to the first end. The sensor tape can further comprise a first portion and a second portion, the first portion having a width substantially the same as the first width, the second portion having a width substantially the same as the second width. The first portion can transition to the second portion in a step-like change. The sensor tape can comprise a sloped transition between the first portion and the second portion. The first and second portions can have substantially the same length.

In some embodiments, a sensor assembly for measuring characteristics of the medium is disclosed. The sensor assembly can comprise a sensor having a detector arm and a connector arm, the detector arm and the connector arm forming an L-shape, and a sensor tape configured to position and secure the sensor to a surface of the medium, the sensor tape having a first end with a first width, a second end with a second width, the second width greater than the first width, the sensor tape further having a flexible tape portion between the first and second ends, the tape portion having an adhesive surface and a non-adhesive surface, and the sensor tape substantially covering the detector arm. The detector arm can comprise an emitter and a detector. The second end of the sensor tape can be closer to the sensor than the first end of the sensor tape. The first end of the sensor tape is closer to the sensor than the second end of the sensor tape. The sensor tape can be tapered such that a width of the tape decreases gradually from the second end to the first end. The sensor tape can further comprise a first portion and a second portion, the first portion having a width substantially the same as the first width, the second portion having a width substantially the same as the second width. The first portion can transition to the second portion in a step-like change. The sensor tape can comprise a sloped transition between the first portion and the second portion. The first and second portions can have substantially the same length.

In some embodiments, a sensor tape for positioning and securing a noninvasive L-shaped sensor to a surface of a medium for measuring characteristics of the medium is disclosed, the L-shaped sensor comprising a detector arm and a connector arm, the detector and connector arms being perpendicular to each other and forming a substantially L-shape, the detector arm comprising an optical emitter and an optical detector. The sensor assembly can comprise a first portion of flexible tape having an adhesive surface and a non-adhesive surface, the first portion having first and second ends, the adhesive surface of the first portion configured to cover the detector arm of the L-shaped sensor and attach to a measurement site, the first portion configured to be substantially parallel to the detector arm; and a second portion of flexible tape having an adhesive surface and a non-adhesive surface, the second portion having first and second ends, the adhesive surface of the second portion configured to attach to a measurement site; wherein the first end of the first portion is connected to the second portion between the first and second ends of the second portion such that the first and second portions are configured to independently wrap around a measurement site. The optical emitter of the detector arm can be configured to be at or near the first end of the first portion and the optical detector is configured to be between the first and second ends of the first portion. The first portion can be longer than the second portion such that the second end of the first portion extends beyond the second end of the second portion. The first and second portions can form an integral piece of sensor tape. The second portion can be configured to cover a portion of the connector arm of the L-shaped sensor. The first and second portions can be mechanically decoupled. The sensor tape can be configured to be placed across a joint of a digit such that the first and second portions are placed on opposite sides of the joint.

All of these embodiments are intended to be within the scope of the disclosure herein. These and other embodiments will become readily apparent to those skilled in the art from the following detailed description having reference to the attached figures, the disclosure not being limited to any particular disclosed embodiment(s).

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1 illustrates a perspective view of an embodiment of a patient monitor system according to the disclosure.

FIG. 2 illustrates a top view of an embodiment of a sensor assembly including an L-shaped sensor and a sensor tape.

FIG. 3 illustrates a top view of an embodiment of a sensor tape.

FIG. 4A illustrates a top view of an embodiment of a sensor assembly including the L-shaped sensor of FIG. 2 and the sensor tape of FIG. 3.

FIG. 4B illustrates a top view of an embodiment of a sensor assembly including the L-shaped sensor of FIG. 2 and the sensor tape of FIG. 3.

FIG. 5A illustrates a top view of an embodiment of a sensor assembly including the L-shaped sensor of FIG. 2 and the sensor tape of FIG. 3.

FIG. 5B illustrates a top view of an embodiment of a sensor assembly including the L-shaped sensor of FIG. 2 and the sensor tape of FIG. 3.

FIG. 6 illustrates a top view of an embodiment of a sensor tape.

FIG. 7A illustrates a top view of an embodiment of a sensor assembly including the L-shaped sensor of FIG. 2 and the sensor tape of FIG. 6.

FIG. 7B illustrates a top view of an embodiment of a sensor assembly including the L-shaped sensor of FIG. 2 and the sensor tape of FIG. 6.

FIG. 8A illustrates a top view of an embodiment of a sensor assembly including the L-shaped sensor of FIG. 2 and the sensor tape of FIG. 6.

FIG. 8B illustrates a top view of an embodiment of a sensor assembly including the L-shaped sensor of FIG. 2 and the sensor tape of FIG. 6.

FIG. 9 illustrates a top view of an embodiment of a sensor tape.

FIGS. 10A-B illustrate top and back perspective views of an embodiment of a sensor assembly including an L-shaped sensor and the sensor tape of FIG. 9.

FIG. 10C illustrates a top view of an embodiment of a sensor assembly including an L-shaped sensor and the sensor tape of FIG. 9.

FIGS. 11A-B illustrate top and front perspective views of an embodiment of the sensor assembly of FIGS. 10A-B connected to a sensor cable.

FIG. 12 illustrates a top view of an embodiment of a sensor tape.

FIGS. 13A-B illustrate top and back perspective views of an embodiment of a sensor assembly including an L-shaped sensor and the sensor tape of FIG. 12.

FIG. 13C illustrates a top view of an embodiment of a sensor assembly including an L-shaped sensor and the sensor tape of FIG. 12.

FIGS. 14A-B illustrate top and front perspective views of an embodiment of the sensor assembly of FIGS. 13A-B connected to a sensor cable.

FIG. 15 illustrates a top view of an embodiment of a sensor assembly including an L-shaped sensor and a two-piece staggered sensor tape.

FIGS. 16A-C illustrate methods of manufacturing the sensor tapes of FIGS. 3, 6, and 9.

### DETAILED DESCRIPTION

Although certain embodiments and examples are described below, those of skill in the art will appreciate that the disclosure extends beyond the specifically disclosed embodiments and/or uses and obvious modifications and equivalents thereof. Thus, it is intended that the scope of the disclosure herein disclosed should not be limited by any particular embodiments described below.

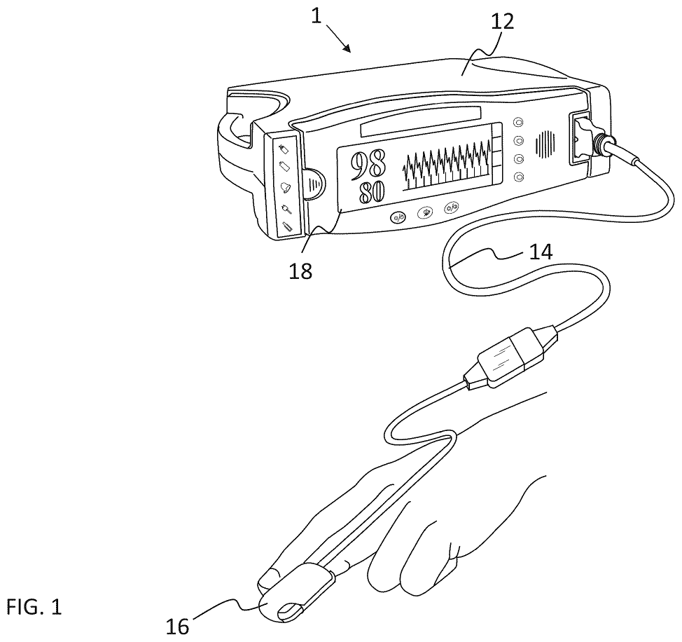

Turning to FIG. 1, an embodiment of a multi-parameter patient monitor system 1 is illustrated. The patient monitor system 1 includes a patient monitor 12 attached to a sensor 16 by a cable 14. The sensor can monitor various physiological data of a patient and send signals indicative of the parameters to the patient monitor 12 for processing. The patient monitor can include a display 18 that is capable of displaying readings of various monitored patient parameters, including one or more graphs. The display 18 may be a liquid crystal display (LCD), a cathode ray tube (CRT), a plasma screen, a Light Emitting Diode (LED) screen, Organic Light Emitting Diode (OLED) screen, or any other suitable display. The patient monitor system 1 may monitor oxygen saturation (SpO₂), perfusion index (PI), pulse rate (PR), hemoglobin count, and other parameters described above. Typically, the patient monitor 12 can also include user control interfaces and a speaker for audible alerts. The patient monitor 12 can also include inputs from other devices, such as, an EKG machine, an ECG machine, a respirator, a ventilator, a blood pressure monitor, a capnograph, combinations of the same, or the like. The sensor 16 can be attached to a measurement site with an attachment mechanism. Non-limiting examples of a measurement site can include a fingertip, arm, leg, or foot of a patient, such as a neonatal patient. The attachment mechanism can be disposable, including, for example, adhesive tapes, hook and loop, magnets or other disposable attachments as described herein.

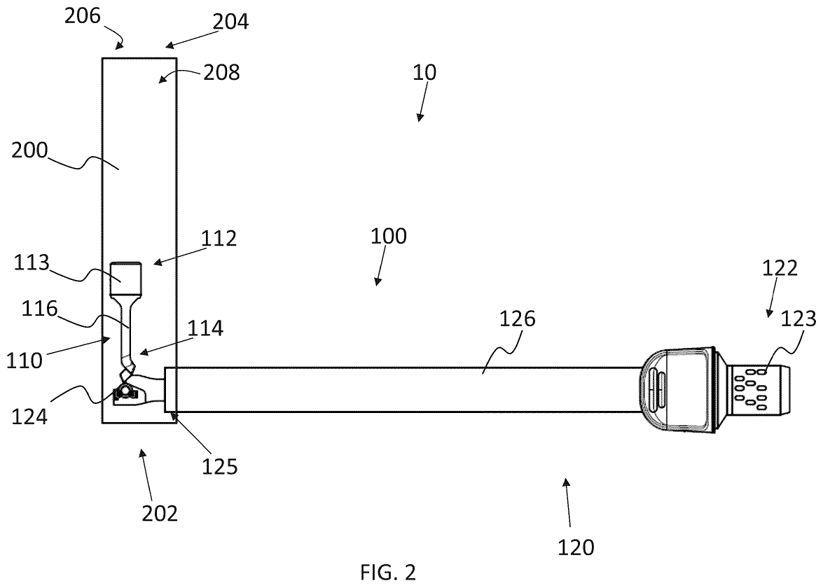

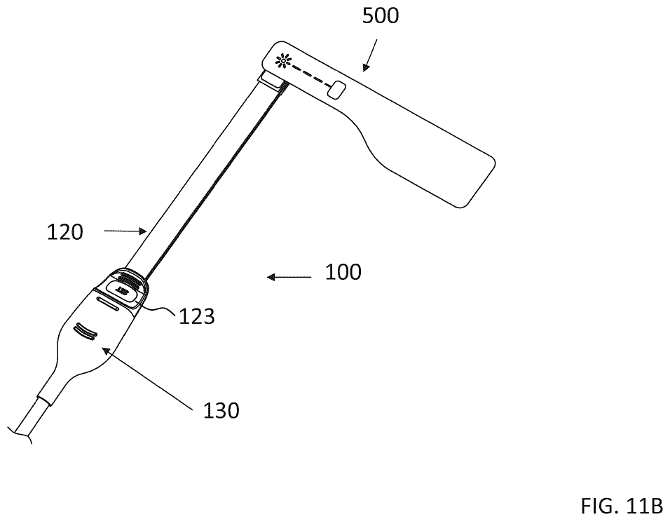

FIG. 2 shows a top view of an embodiment of a sensor assembly 10 including an L-shaped sensor 100 and a sensor tape 200. The sensor 100 can have a detector arm 110 and a connector arm 120 forming a substantially L-shape. The detector arm 110 can have a free end 112 on and a fixed end 114. The detector arm 110 can have a detector 113 at or near the free end 112. The detector arm 110 can have an emitter 124 at or near the fixed end 114. The emitter 124 can be located anywhere between the free and fixed ends 112, 114. The detector 113 and the emitter 124 locations can be swapped so that the emitter 124 is located at or near the free end 112 and the detector 113 is located at or near the fixed end 114. The detector 112 and the fixed end 114 can be connected by a neck portion 116. The neck portion 116 may vary in length depending on the patient's anatomy so that the detector 112 and an emitter 124 can be positioned on opposite sides of the patient's anatomy. In some embodiments, the emitter 124 can include one or more LEDs. In some embodiments, the detector 112 can include one or more photodetectors. The fixed end 114 can be connected to the connector arm 120. The connector arm 120 can have a cable connector 123 on a free end 122. The connector arm 120 can have a fixed end 125 opposite the free end 122 along a length of the connector arm 120 for connecting to the fixed end 114 of the detector arm 110. The connector arm 120 can also include a flexible foam strip 126 extending between the cable connector 123 and the emitter 124. The cable connector 122, the emitter 124, and the detector 112 can be electrically connected to form a portion of an electrical circuit. The flexible foam strip 126 can protect the electrical circuit. The electrical circuit can be configured to attach to other electrical components, such as a resistor and/or an electrically erasable programmable read-only memory (“EEPROM”), which are not shown in the figures for clarity. The cable connector 123 can be operably coupled to a sensor cable (see FIGS. 11A-B and 14A-B), which can be plugged into a variety of patient monitors, such as the patient monitor 12 of FIG. 1, or pulse oximeters or any other multi-parameter monitors for providing noninvasive physiological measurements. Methods of manufacturing the L-shaped sensor is not limiting. In some embodiments, the L-shaped sensor 100 can be manufactured as a straight arm and be folded into the L-shape. In other embodiments, the fixed end 114 of the detector arm 110 and the fixed end 125 of the connector arm 120 can be attached mechanically, welded or affixed using adhesives. Additional details regarding the L-shaped sensor and other features can be found in U.S. application Ser. No. 15/017,505, filed Feb. 5, 2016, which is hereby incorporated by reference in its entirety and should be considered a part of this specification. The L-shaped sensor can be used for infant patients as the flexible connector arm 120 can give a caregiver more flexibility for connecting the sensor to various locations on an infant or neonatal patient. Although the present disclosure is described mainly with reference to the L-shaped sensor, the embodiments of sensor tapes described herein are not limited to being used with an L-shaped sensor, but are applicable to any suitable type of sensors. For example, the tapes disclosed herein can be used for securing sensors onto a fingertip or arm of an adult patient.

With continued reference to FIG. 2, the sensor tape 200 can cover the detector arm 110 of the L-shaped sensor 100. The sensor tape 200 can have a sensor end 202 and a free end 204. The sensor end 202 can be proximate the emitter 124. The sensor tape 200 can extend along a length of the detector arm 110, past the detector 113, and terminate at the free end 204. The sensor tape 200 can be substantially longer than the detector arm 110 so as to wrap around the patient's anatomy in more than one loop. The length of the sensor tape 200 can depend on the dimension of the patient's anatomy to which the sensor tape 200 can be applied. The sensor tape 200 can have an adhesive side 206 and a non-adhesive side 208. Materials for making the adhesive side 206 and the non-adhesive side 208 are not limiting. In some embodiments, the detector arm 110 can be sandwiched between the adhesive side 206 and the non-adhesive side 208. In some embodiments, the detector arm 110 can be positioned beneath or immediately adjacent to the adhesive side 206. The sensor tape 200 can have a rectangular shape with a substantially uniform first width and a length. During use, the neck portion 116 of the detector arm can be wrapped around a patient's anatomy, such as a foot, a hand, a finger, or a toe, so that the emitter 124 and the detector 112 are on opposite sides of the patient's anatomy. For example, the emitter 124 can be on a patient's palm and the detector 112 can be on a back of the same hand opposite the emitter 124. After the emitter 124 and the detector 112 have been positioned, the sensor tape 200 can continue to be wrapped around the patient's anatomy from the sensor end 202 to the free end 204. An initial length of the adhesive side 206 of the sensor tape 200 can directly contact the patient's skin, resulting in a contact area. After the sensor tape 200 has made one loop around the patient's anatomy, a remaining length of the adhesive side 206 of the sensor tape 200 can contact substantially the non-adhesive side 208 of the tape instead of the patient's skin. The sensor tape 200 advantageously reduces motion-induced noise by firmly positioning and securing the emitter 124 and the detector 112 to the patient's skin, thereby minimizing movements of the sensor 100 relative to the patient due to patient's movement. Bonding formed between the remaining length of the adhesive side 206 and the non-adhesive side 208 can prevent the sensor tape 200 from loosening, thereby facilitating the secure attachment of the sensor tape 200 with the emitter 124 and the detector 113 to the patient at or near the measurement site. The L-shaped sensor 100 is typically attached to the measurement site such that the connector arm 120 extends along the patient's anatomy, such as the patient's finger, hand, toe, foot, arm, or leg. The patient's anatomy can provide support to the connector arm 120 or protect the connector arm 120 from being pulled during use of the sensor. In some instances, the caregiver or user can attach the L-shaped sensor 100 to the measurement site such that the connector arm 120 extends away from the patient's anatomy. In these instances, the connector arm 120 can be tangling from the measurement site and prone to pulling. Pulling on the connector arm 120 can cause the sensor 100 and the sensor tape 200 be yanked away from the patient's skin.

Various embodiments of sensor tapes that can improve securement of the L-shaped sensor 100 to the measurement site will now be described. The improved sensor tapes described herein can minimize or eliminate sliding between the detector arm 110 of the sensor 100 and the patient's skin during use of the sensor 100. The sliding can be caused by the patient's movement or due to pulling on the connector arm 120 or the sensor cable. The improved sensor tapes described herein can provide sufficient bonding between the tape and the patient's skin such that even when the L-shaped sensor 100 is attached with the connector arm 120 extending away from the patient's anatomy, the improved sensor tape and the sensor 100 can stay attached to the patient's skin.

### Tapered Sensor Tapes

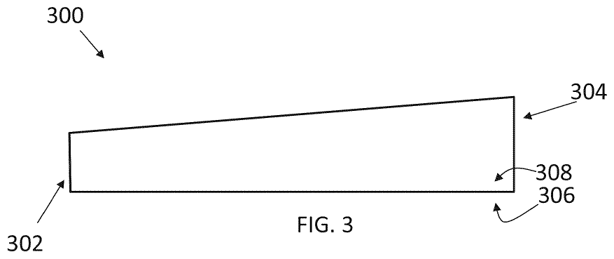

FIG. 3 shows a top view of a tapered sensor tape 300. The sensor tape 300 can have features of the sensor tape 200 except as described below. Accordingly, features of the sensor tape 300 can be incorporated into features of the sensor tape 200 and features of the sensor tape 200 can be incorporated into features of the sensor tape 300. The sensor tape 300 can have a first end 302 and a second end 304. The sensor tape 300 can have a first width at the first end 302. The sensor tape 300 can have a second width at the second end 304. As shown in FIG. 3, the second width is greater than the first width. The first width of the sensor tape 300 can be substantially the same as the first width of the sensor tape 200 as shown in FIG. 2. Accordingly, the sensor tape 300 tapers, for example, gradually tapers, from the second end 304 to the first end 302. The geometry between the first end 302 and second end 304 is not limiting. For example, the sensor tape 300 can have wavy edges instead of straight edges on any of the four sides. The sensor tape 300 can also have an adhesive side 306 and a non-adhesive side 308.

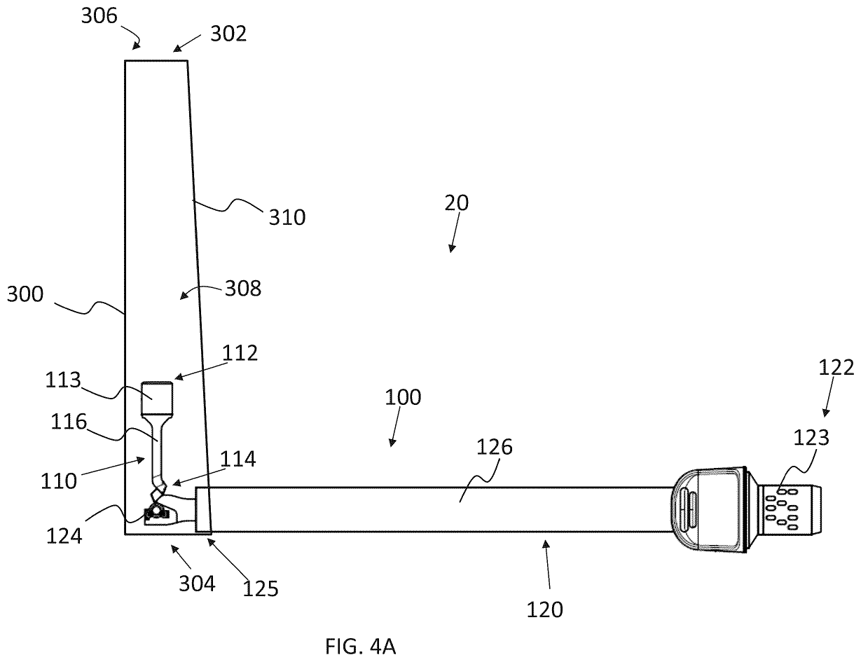

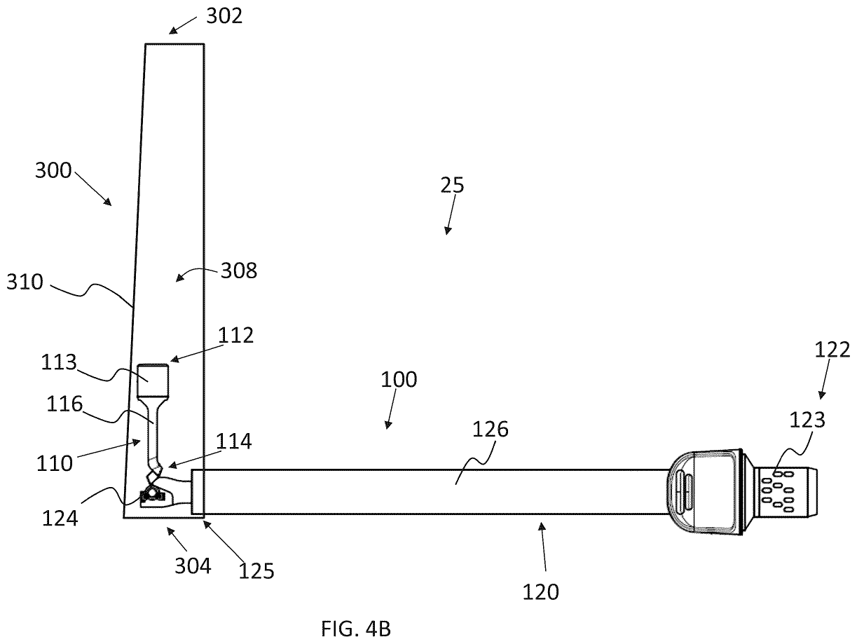

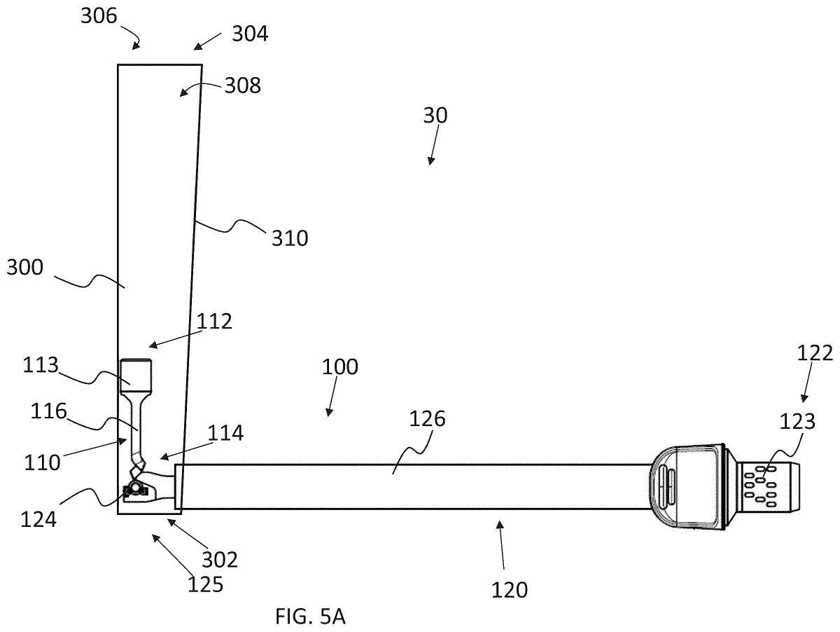

FIGS. 4A-B illustrate embodiments of sensor assembly 20, 25 including the L-shaped sensor 100 and the tapered sensor tape 300. As shown in FIGS. 4A-B, the second end 304 of the sensor tape 300 can be proximate the emitter 124. The sensor tap 300 can extend along a length of the detector arm 110, past the detector 113, and terminate at the first end 302. The sensor tape 300 can be substantially parallel to the detector arm 110. The second width and a length of the sensor tape 300 are sufficient to cover the detector arm 110. The sensor tape 300 can be substantially longer than the length of the detector arm 110. In FIG. 4A, the tapered side 310 of the sensor tape 300 can be closer to the connector arm 120 than the non-tapered opposite side. In FIG. 4B, the tapered side 310 can be further away from the connector arm 120 than the non-tapered opposite side. During use, the sensor tape 300 can be wrapped around the patient's anatomy from the second end 304 to the first end 302. An initial length of the adhesive side 306 of the sensor tape 300 can directly contact the patient's skin, resulting in a contact area. The contact area between the sensor tape 300 and the patient's skin is greater than the contact area between the sensor tape 200 and the patient's skin, because the sensor tape 300 is wider near the fixed end 114 of the detector arm 110 of the sensor 100 in the sensor assembly 20, 25 than the sensor tape 200 in the sensor assembly 10. The sensor tape 300 in the sensor assembly 20, 25 can advantageously provide greater contact area and thus better securement between the sensor assembly and the patient's skin, thereby further minimizing movements of the sensor 100 relative to the patient's skin. Further, as described above, after the sensor tape 300 has made one loop around the patient's anatomy, a remaining length of the adhesive side 306 can contact substantially the non-adhesive side 308 of the tape instead of the patient's skin. Accordingly, the sensor tape 300 can provide better securement of the L-shaped sensor 100 by providing a greater contact area between the adhesive side 306 of the sensor tape 300 with the patient skin than the sensor tape 200, but without requiring a significant increase in use of tape materials due to the tapering of the sensor tape 300.

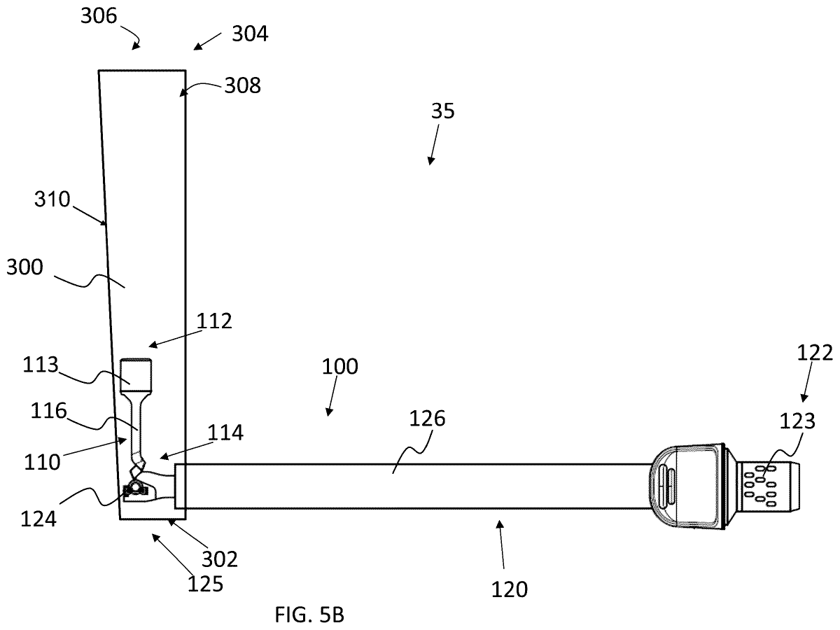

FIGS. 5A-B illustrate embodiments of sensor assembly 30, 35 including the L-shaped sensor 100 and the tapered sensor tape 300. As show in FIGS. 5A-B, the first end 302 of the sensor tape 300 can be proximate the emitter 124. The sensor tape 300 can extend along a length of the detector arm 110, past the detector 112, and terminate at the second end 304. The sensor tape 300 can be substantially parallel to the detector arm 110. The first width of the sensor tape 300 can be sufficient to cover the detector arm 110. In FIG. 5A, the tapered side 310 of the sensor tape 300 can be closer to the connector arm 120 than the non-tapered opposite side. In FIG. 5B, the tapered side 310 can be further away from the connector arm 120 than the non-tapered opposite side. During use, after the sensor tape 300 has made a first loop around the patient's anatomy, a remaining length of the adhesive side 306 can contact partially the non-adhesive side 308 of the first loop and partially the patient's skin because the sensor tape 300 becomes increasing wider from the fixed end 114 to the free end 112 of the detector arm 110. Specifically, the sensor tape 300 is narrower near the emitter 124 and gradually widens toward the detector 112. As a result, after each loop of the sensor tape 300 around the patient's anatomy, the adhesive side 306 of the sensor tape 300 is wider than the non-adhesive side 308 of the previous loop. The wider adhesive side 306 can then contact the skin not covered by the non-adhesive side 308 of the previous loop. The total contact area between the sensor tape 300 of the sensor assembly 30, 35 and the patient's skin is thus higher than the contact area between the sensor tape 200 and the patient's skin. In addition, the narrow first end 302 of the tape 300 can be easier to place on the finger to align the emitter 124 and the detector 113 before the wider second end 304 can wrap the detector arm 110 and the narrower part of the sensor tape 300 in place. The wider second end 304 can attach a portion of the connector arm 120 to the patient's skin. The sensor assembly 30, 35 can therefore better position and secure the sensor 100 to the patient's skin than the sensor tape 200, while not requiring a significant increase in use of tape materials due to the tapering of the sensor tape 300.

### Stepped Sensor Tapes

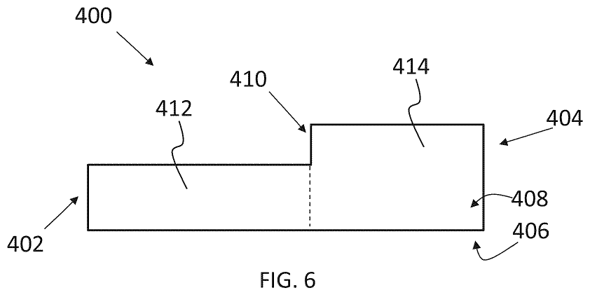

FIG. 6 illustrates a top view of a stepped sensor tape 400. The sensor tape 400 can have features of the sensor tapes 200, 300 except as described below. Accordingly, features of the sensor tape 400 can be incorporated into features of the sensor tapes 200, 300 and features of the sensor tapes 200, 300 can be incorporated into features of the sensor tape 400. The sensor tape 400 can have a first end 402 and a second end 404. The sensor tape 400 can have a first width at the first end 402. The sensor tape 400 can have a second width at the second end 404. As show in FIG. 6, the second width is greater than the first width. The first width of the sensor tape 400 can be substantially the same as the first widths of the sensor tapes 200, 300. The sensor tape 400 can transition from the first width to the second width in a step-like change 410. The step-like transition 410 can be at a location between the first end 402 and the second end 404. The step-like transition 410 can separate the sensor tape 400 into a first portion 412 and a second portion 414. The exact geometries of the first portion 412 and the second portion 414 are not limiting. For example, at least one of the first portion 412 and the second portion 414 can have wavy edges on any sides. The step-like change 410 can be on one side or both sides along the length of the sensor tape 400. The sensor tape 400 can have an adhesive side 406 and a non-adhesive side 408.

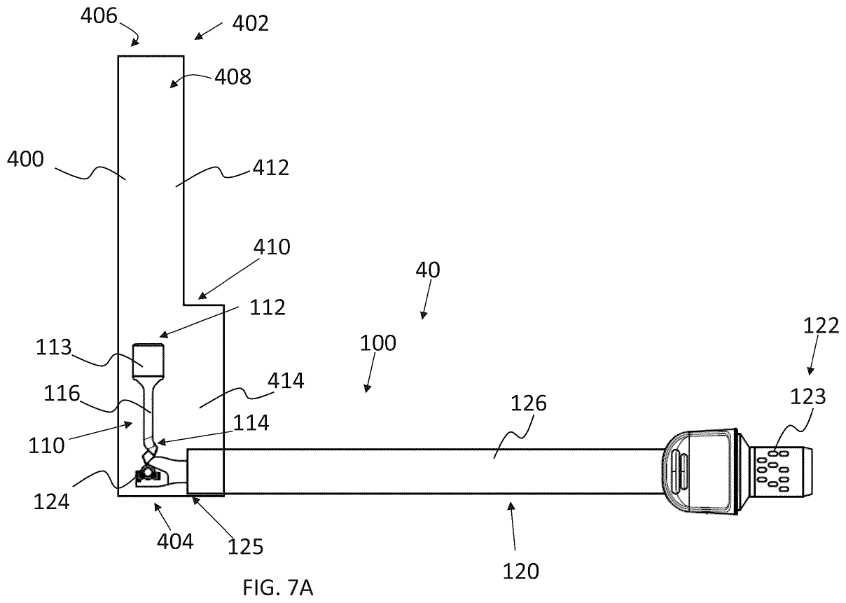

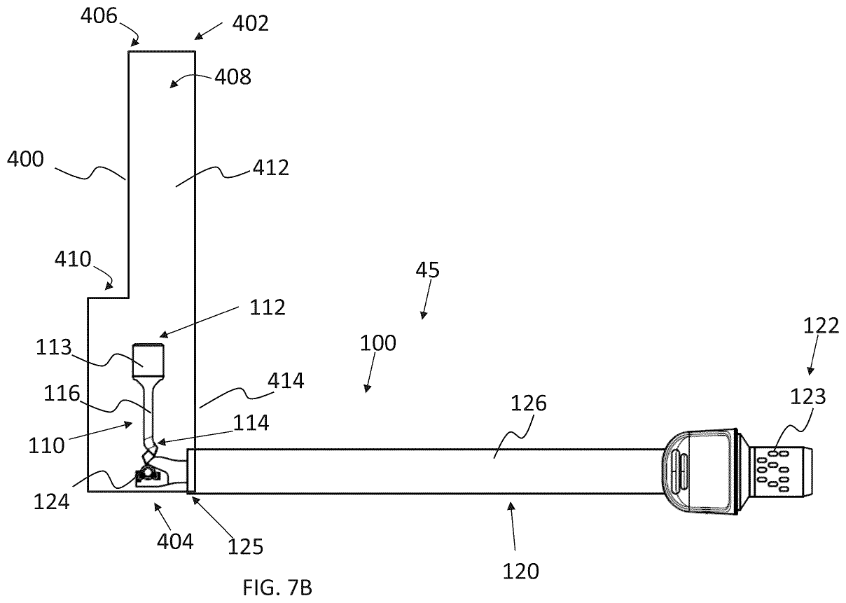

FIGS. 7A-B illustrate embodiments of sensor assembly 40, 45 including the L-shaped sensor 100 and the stepped sensor tape 400. As shown in FIGS. 7A-B, the second end 404 of the sensor tape 400 can be proximate the emitter 124. The sensor tap 400 can extend along a length of the detector arm 110, past the detector 113, and terminate at the first end 402. The sensor tape 400 can be substantially parallel to the detector arm 110. The second width and a length of the sensor tape 400 can be sufficient to cover the detector arm 110. In FIG. 7A, the step-like transition 410 of the sensor tape 400 can be closer to the connector arm 120 than the opposite side without the step-like transition. In FIG. 7B, the step-like transition 410 can be further away from the connector arm 120 than the opposite side without the step-like transition. During use, the sensor tape 400 can then be wrapped around the patient's anatomy from the second end 404 to the first end 402. An initial length of the adhesive side 406 of the sensor tape 400, which can include the second portion 414, can directly contact the patient's skin, resulting in a contact area. The contact area between the adhesive side 406 of the sensor tape 400 and the patient's skin is greater than the contact area between the sensor tape 200 and the patient's skin. This is because the sensor tape 400 is wider near the fixed end 114 of the detector arm 110 of the sensor 100 in the sensor assembly 40, 45 than the sensor tape 200 in the sensor assembly 10. The sensor tape 400 in the sensor assembly 40 can advantageously provide greater contact area and thus better securement between the sensor assembly and the patient's skin, thereby minimizing movements of the sensor 100 relative to the patient's skin. Further, after the second portion 414 of the sensor tape 400 has made a first loop around the patient's anatomy, a remaining length of the adhesive side 406 can contact substantially the non-adhesive side 408 of the first loop instead of the patient's skin. The contact between the remaining length of the adhesive side 406 with the non-adhesive side 408 of the first loop can prevent the sensor tape 400 from loosening. Accordingly, the sensor tape 400 of the sensor assembly 40, 45 can provide better securement of the L-shaped sensor 100 by providing a greater contact area between the adhesive side 406 of the sensor tape 400 with the patient skin than the sensor tape 200, but without requiring a significant increase in use of tape materials due to the first portion 412 being narrower than the second portion 414.

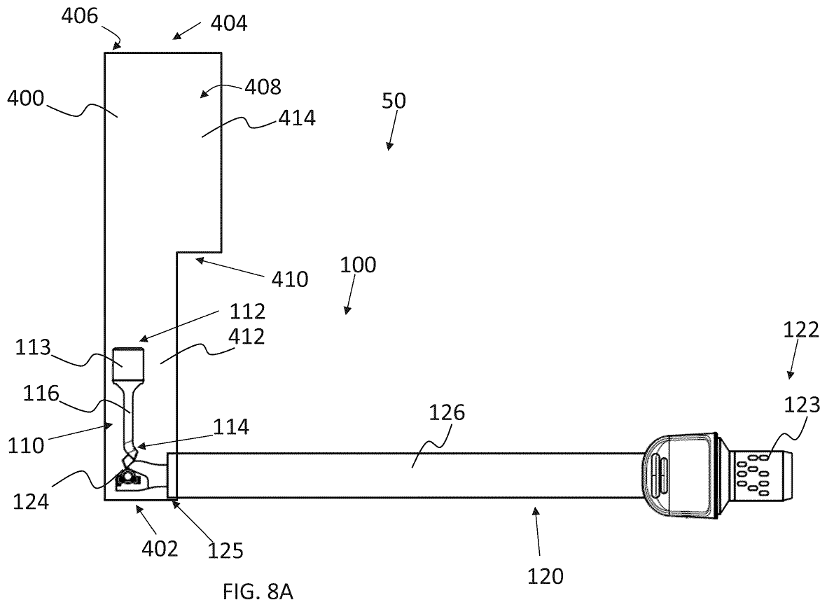

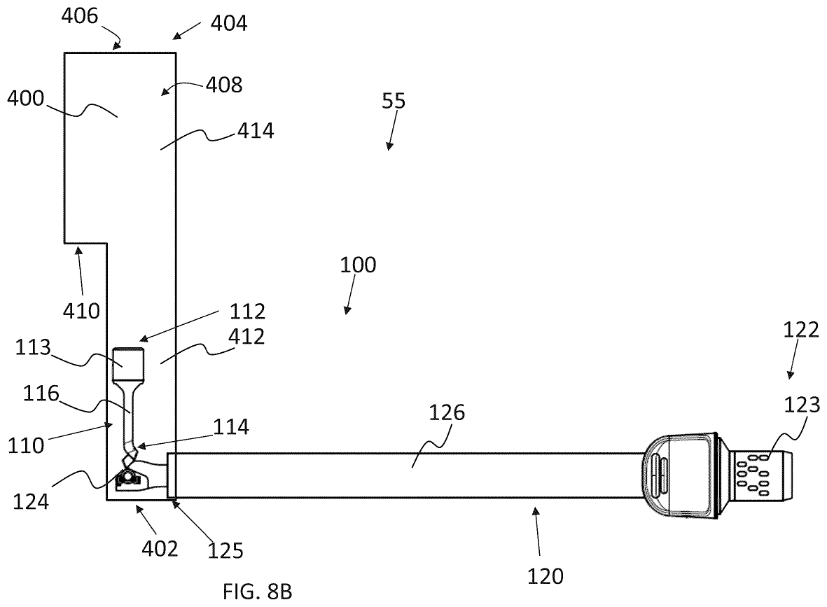

FIGS. 8A-B illustrate embodiments of a sensor assembly 50, 55 including the L-shaped sensor 100 and the stepped sensor tape 400. As show in FIGS. 8A-B, the first end 402 of the sensor tape 400 can be proximate the emitter 124. The sensor tape 400 can extend along a length of the detector arm 110, past the detector 113, and terminate at the second end 404. The sensor tape 400 can be substantially parallel to the detector arm 110. The first width of the sensor tape 400 can be sufficient to cover the detector arm 110. In FIG. 8A, the step-like transition 410 of the sensor tape 400 can be closer to the connector arm 120 than the opposite side without the step-like transition. In FIG. 8B, the step-like transition 410 can be further away from the connector arm 120 than the opposite side without the step-like transition. During use, after the narrow first portion 412 of the sensor tape 400 runs out, the adhesive side 406 of the wide second portion 414 can contact partially the non-adhesive side 408 of the narrow first portion 412 and partially the patient's skin not covered by the non-adhesive side 408 of the narrow first portion 412. The total contact area between the sensor tape 400 of the sensor assembly 50, 55 and the patient's skin is higher than the contact area between the sensor tape 200 and the patient's skin. In addition, the narrow first portion 412 can be easier to place on the finger to align the emitter 124 and the detector 113 before the wider second portion 414 can wrap the detector arm 110 and the narrow first portion 412 in place. The wider second portion 414 can attach a portion of the connector arm 120 to the patient's skin. The sensor assembly 50, 55 can therefore better secure the sensor 100 to the patient's skin than the sensor tape 200 and without requiring a significant increase in use of tape materials due to the first portion 412 of the sensor tape 400 of being narrower than the second portion 414.

In some embodiments, the first portion 412 and the second portion 414 of the sensor tape 400 can have substantially equal lengths. In some embodiments, the first portion 412 and the second portion 414 can have different lengths. Ratio of the respective lengths of the first portion 412 and the second portion 414 is not limiting. For example, the first portion 412 can have a length sufficient for making at least one loop around a patient's anatomy. The second portion 414 can have a length sufficient for making at least one loop around a patient's anatomy.

### Sloped Sensor Tapes

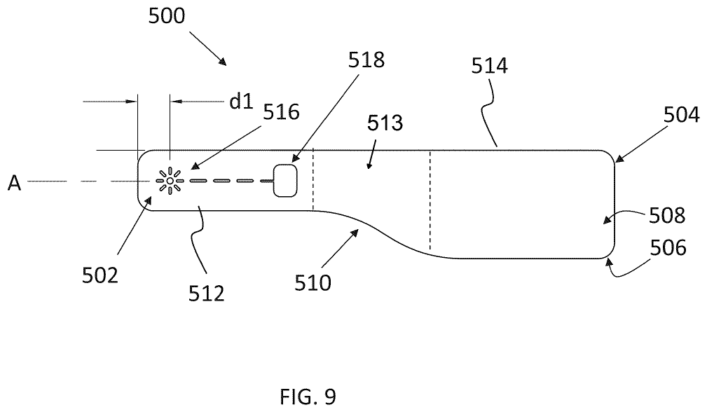

FIG. 9 illustrates a top view of a sloped sensor tape 500. The sensor tape 500 can have features of the sensor tapes 200, 300, 400 except as described below. Accordingly, features of the sensor tape 500 can be incorporated into features of the sensor tapes 200, 300, 400 and features of the sensor tapes 200, 300, 400 can be incorporated into features of the sensor tape 500. The sensor tape 500 can have a first end 502 and a second end 504. The sensor tape 500 can have a first width at the first end 502. The sensor tape 500 can have a second width at the second end 504. As show in FIG. 9, the second width is greater than the first width. The first width of the sensor tape 500 can be substantially the same as the first widths of the sensor tapes 200, 300, 400. The sensor tape 500 can transition from the first width to the second width in a slope 510. The slope 510 can be at a location between the first end 502 and the second end 504. The slope 510 can separate the sensor tape 500 into a first portion 512, a second portion 514, and a transition portion 513. The exact geometries of the first portion 512, the second portion 514, and the transition portion 513 are not limiting. For example, at least one of the first portion 512, the second portion 514, and the transition portion can have wavy edges along any sides. The transition portion 513 can have a straight-line slope or a curved slope. The transition portion 513 can have a slope on one side or both sides along the length of the sensor tape 500. The sensor tape 500 can have an adhesive side 506 and a non-adhesive side 508.

With continued reference to FIG. 9, the non-adhesive side 508 of the sensor tape 500 can have alignment indicators 516, 518. The indicator 516 can be aligned with the emitter 124 of the L-shaped sensor 100. The indicator 518 can be aligned with the detector 113 of the L-shaped sensor 100. The alignment indicators 516, 518 can facilitate accurate placement of the detector arm 110 onto the sensor tape 500. For example, both indicators 516, 518 can be centered along a central axis or midline “A” of the narrower first portion 512, as shown in FIG. 9, or along a central axis or midline of the wider second portion 514. A center of the indicator 516 can be a distance d1 from the first end 502 of the sensor tape 500. The alignment indicators 516, 518 can also provide visual aid to a user or a caregiver to ensure that the emitter 124 and the detector 113 are aligned during securement of the sensor 100 to the measurement site with the sensor tape 500.

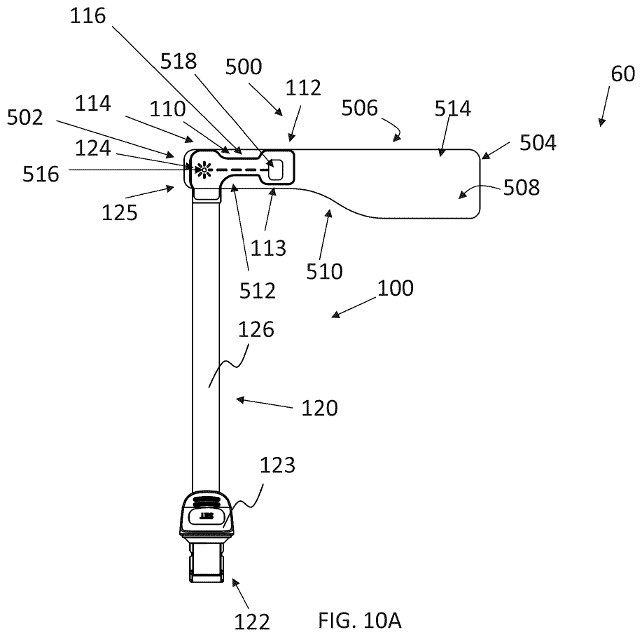

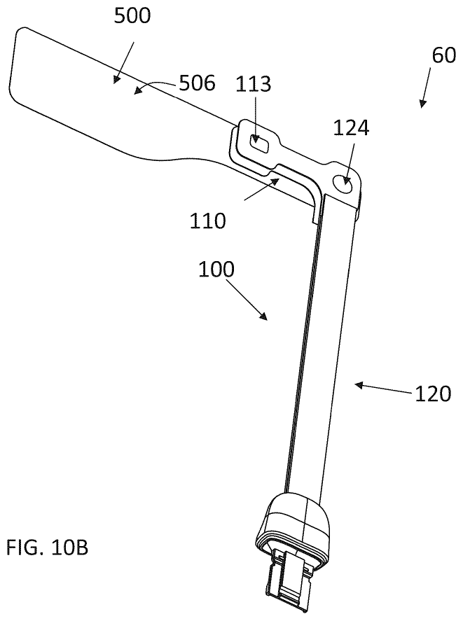

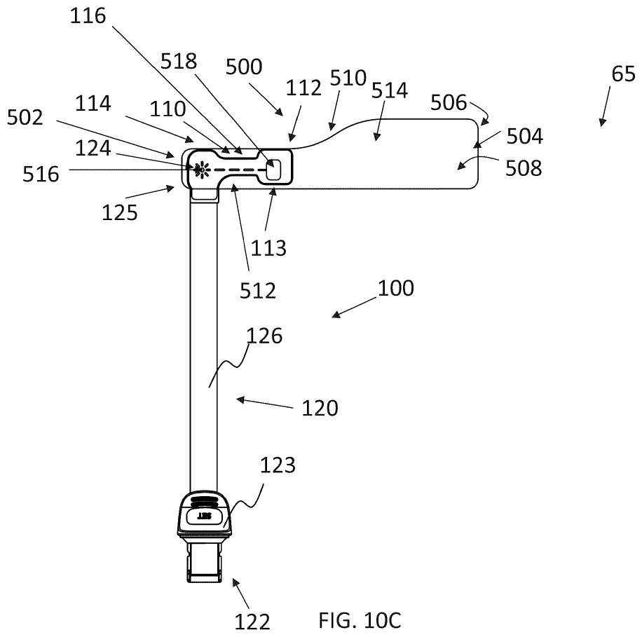

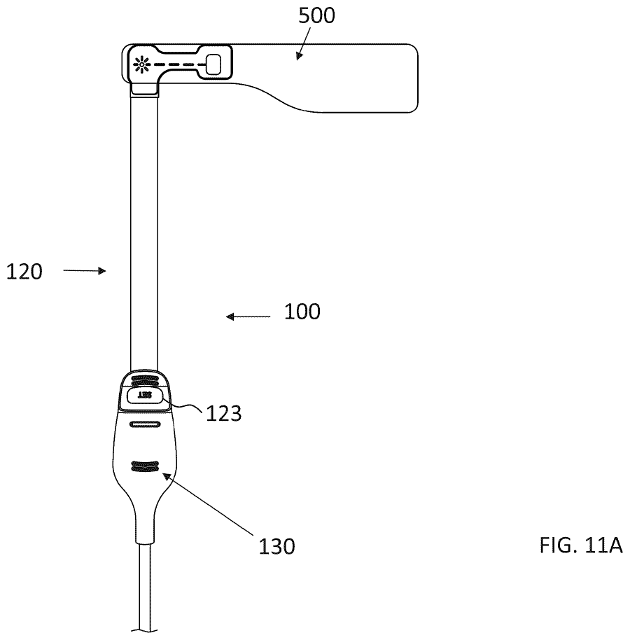

FIGS. 10A-C illustrate embodiments of a sensor assembly 60, 65 including the L-shaped sensor 100 and the sloped sensor tape 500. FIGS. 11A-B illustrate the sensor assembly 60 including the L-shaped sensor 100 and the sloped sensor tape 500 being connected to a sensor cable 130 at the cable connector 123. As shown in FIGS. 10A-C, the first end 502 of the sensor tape 500 can be proximate the emitter 124. The indicator 516 can be aligned with the emitter 124. The sensor tape 500 can be substantially parallel to the detector arm 110. The sensor tape 500 can extend along the length of the detector arm 110, past the detector 113, and terminate at the second end 504. The first width and a length of the sensor tape 400 can be sufficient to cover the detector arm 110. The indicator 518 can be aligned with the detector 113. In FIGS. 10A-B, the slope 510 of the sensor tape 500 can be closer to the connector arm 120 than the opposite side without the slope. In FIG. 10C, the slope 510 can be further away from the connector arm 120 than the opposite side without the slope. During use, the narrow first portion 512 of the sensor tape 500 can contact the patient's skin at or near the measurement site. As shown in FIGS. 10A-C, the narrow first portion 512 terminates at or near the free end 112 of the detector arm 110. The first portion 512 can cover approximately half a loop around the patient's anatomy. The adhesive side 506 of the increasingly wider transition portion 513 can contact the patient's skin along a portion of or an entire second half of the loop around the patient's anatomy. After the transition portion 513 runs out, the adhesive side 506 of the wider second portion 514 can contact partially the non-adhesive side 508 of the narrow first portion 512 and/or the transition portion 513, and partially the patient's skin not covered by the non-adhesive side 508 of the first portion 512 and/or the transition portion 513. The total contact area between the sensor tape 500 of the sensor assembly 60 and the patient's skin is higher than the contact area between the sensor tape 200 and the patient's skin. In addition, the narrow first portion 512 can be easier to place on the finger to align the emitter 124 and the detector 113 before the wider second portion 514 can wrap the detector arm 110 and the narrow first portion 512 in place. The transition portion 513 and the wider second portion 514 can attach a portion of the connector arm 120 to the patient's skin. The sensor assembly 60 can therefore better secure the sensor 100 to the patient's skin than the sensor tape 200 and without requiring a significant increase in use of tape materials due to the first portion 512 and the transition portion 513 of the sensor tape 500 being narrower than the second portion 514.

The transition portion 513 of the sloped sensor tape 500 can avoid sharp corners of a stepped transition. The transition portion 513 can thus reduce tearing of a sensor tape at or around the sharp corner when applying or removing the sensor tape. As show in FIG. 10B, the detector arm 110 of the sensor 110 is placed beneath or immediately next to the adhesive side 506 of the sensor tape 500. The emitter 124 and the detector 113 can be aligned to the indicators 516, 518 as discussed above to ensure that the detector arm 110 is placed within the boundary of the sensor tape 500. Placing the sensor 100 next to the adhesive side 506 of the sensor tape 500 can allow the sensor and the tape be assembled right before use. The sensor tape 500 can come in a variety of sizes, such as small, medium, and large. The appropriately sized sensor tape 500 can be selected depending on the size of the patient's anatomy. The separability of the sensor tape 500 from the sensor 100 can allow the sensor tape 500 to be disposable so that a new sensor tape 500 with a fresh adhesive side 506 can be used for every measurement site to improve securement of the sensor 100 to the measurement site. The separability of the sensor tape 500 from the sensor 100 can allow the more expensive components, such as the emitter 124, the detector 113, and other electrical components, be reusable. Reusing the more expensive components can reduce cost of replacing the optical sensors.

Similar to the assemblies of the sensor tapes 300, 400 and the L-shaped sensor 100 described above, the sensor tape 500 can be used with the sensor 100 such that the second side 504 is approximate the emitter 124. The indicators can be placed on the second portion 514 and be centered on the central axis or midline of the wider second portion 514.

### Staggered Sensor Tapes

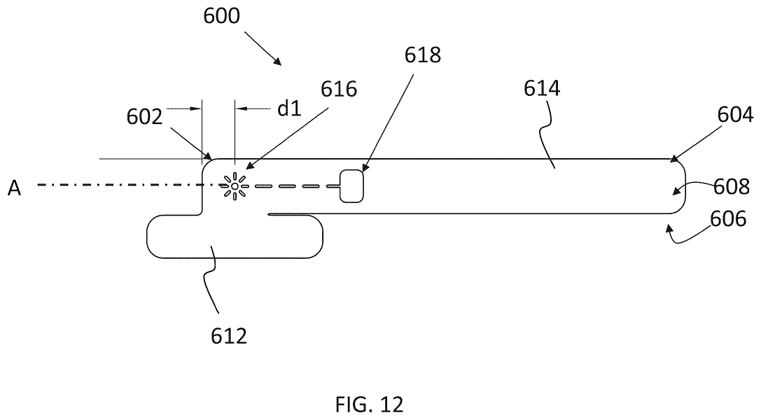

FIG. 12 illustrates a top view of a staggered sensor tape 600. The sensor tape 600 can have features of the sensor tapes 200, 300, 400, 500 except as described below. Accordingly, features of the sensor tape 600 can be incorporated into features of the sensor tapes 200, 300, 400, 500 and features of the sensor tapes 200, 300, 400, 500 can be incorporated into features of the sensor tape 600. The sensor tape 600 can have a first portion 612 and a second portion 614. The first and second portions 612, 614 can be cut from the same piece of tape material. The first and second portions 612, 614 can be an integral sensor tape. The first and second portions 612, 614 can be connected at a first end 602 of the second portion 614 such that sections of the two portions are staggered. The first portion 612 can be substantially centered at the first end 602 of the second portion 614 such that one end of the first portion 612 extends beyond the first end 602 of the second portion 614. In other embodiments, the first portion 612 can have about ⅔ of its length extending beyond the first end 602 of the second portion 614 and the remaining about ⅓ of its length aligned with the second portion 614. The length of the first portion 612 that extends beyond the first end 602 of the second portion 614 is not limiting. The first portion 612 can have a first width. The second portion 614 can have a second width. The second portion 614 can have a second end 604 opposite the first end 602 along a length of the second portion 614. The length of the second portion 614 can be greater than a length of the first portion 612. As show in FIG. 12, the second width is greater than the first width. The first and second widths can be substantially the same. The second width can be smaller than the first width. The second width of the sensor tape 600 can be substantially the same as the first widths of the sensor tapes 200, 300, 400, 500. The exact geometries of the first portion 612 and the second portion 614 are not limiting. For example, at least one of the first portion 512 and the second portion 614 can have straight or wavy edges along any sides.

With continued reference to FIG. 12, the sensor tape 600 can have an adhesive side 606 and a non-adhesive side 608. The non-adhesive side 608 of the sensor tape 600 can have alignment indicators 616, 618. The indicator 616 can be aligned with the emitter 124 of the L-shaped sensor 100. The indicator 618 can be aligned with the detector 113 of the L-shaped sensor 100. The alignment indicators 616, 618 can facilitate accurate placement of the detector arm 110 onto the sensor tape 600. For example, both indicators 616, 618 can be centered along a central axis or midline “A” of the wider second portion 614, as shown in FIG. 12, or along a central axis or midline of the narrow first portion 612. A center of the indicator 616 can be a distance d1 from the first end 602 of the sensor tape 600. The alignment indicators 616, 618 can also provide visual aid to a user or a caregiver to ensure that the emitter 124 and the detector 113 are aligned during securement of the sensor 100 to the measurement site with the sensor tape 600.

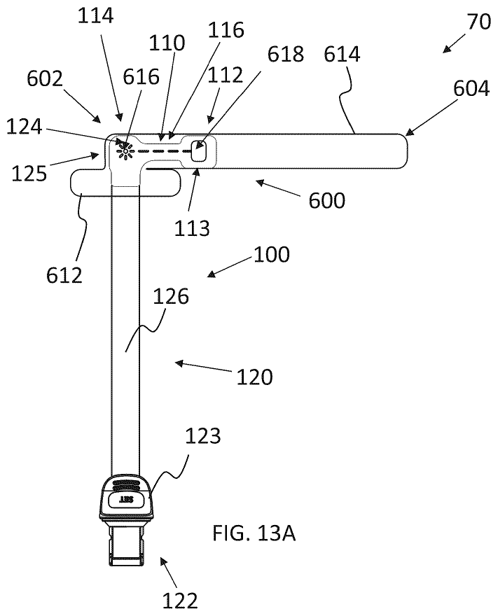

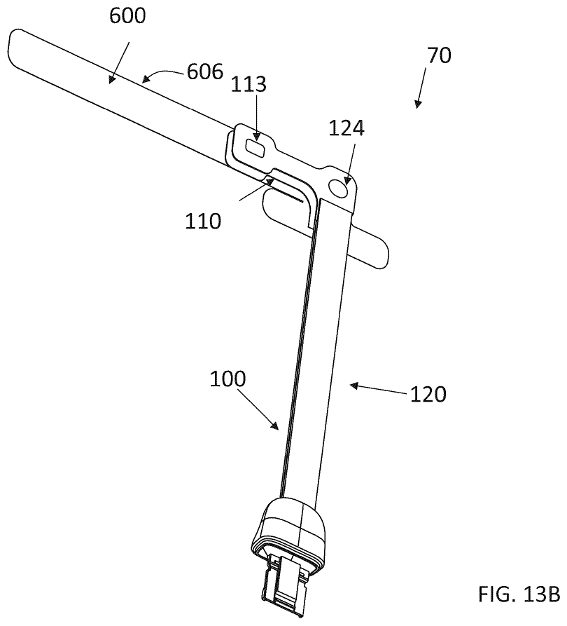

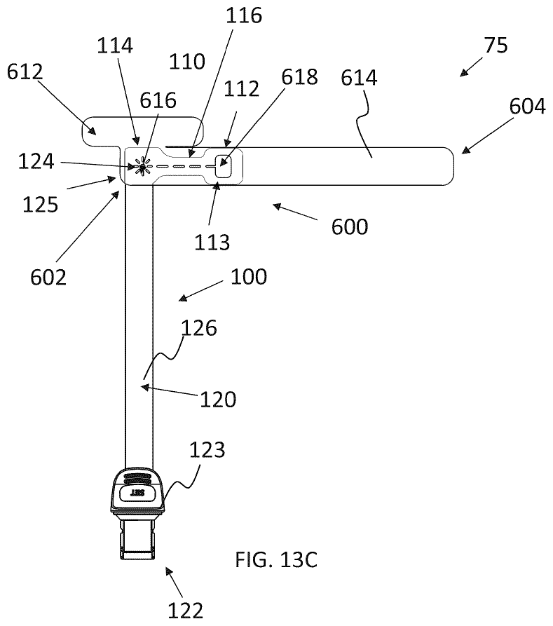

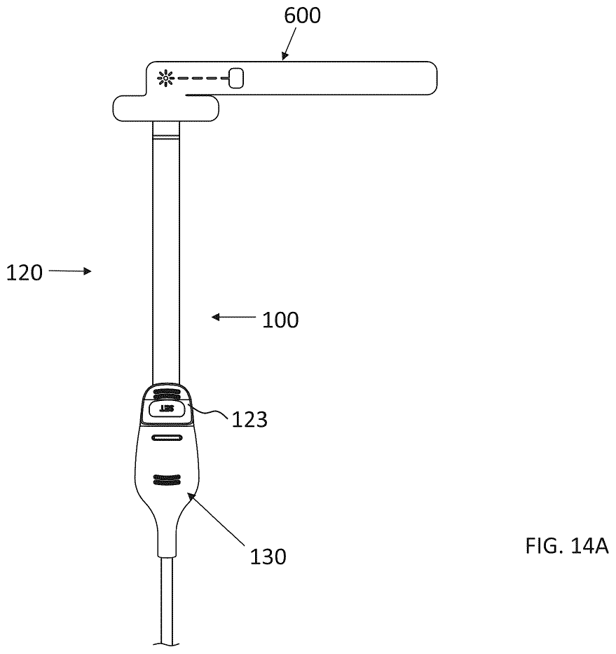

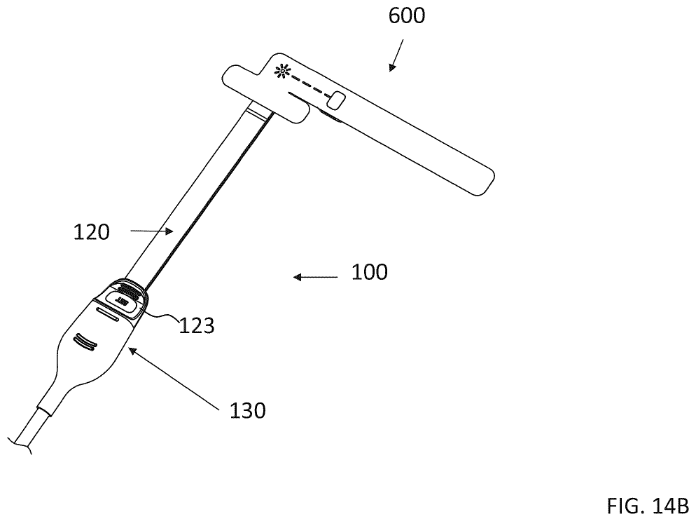

FIGS. 13A-C illustrate embodiments of a sensor assembly 70, 75 including the L-shaped sensor 100 and the staggered sensor tape 600. FIGS. 14A-B illustrate the sensor assembly 70 including the L-shaped sensor 100 and the staggered sensor tape 600 being connected to a sensor cable 130 at the cable connector 123. As shown in FIGS. 13A-C, the first end 602 of the sensor tape 600 can be proximate the emitter 124. The indicator 616 can be aligned with the emitter 124. The sensor tape 600 can extend along the length of the detector arm 110, past the detector 113, and terminate at the second end 604. The sensor tape 600 can be substantially parallel to the detector arm 110. The second width of the sensor tape 600 can be sufficient to cover the detector arm 110. The indicator 618 can be aligned with the detector 113. In FIGS. 13A-B, the first portion 612 of the sensor tape 500 can be closer to the connector arm 120 than the second portion 614. In FIG. 13C, the first portion 612 can be further away from the connector arm 120 than the second portion 614. During use, the wider second portion 614 and the narrow first portion 612 of the sensor tape 600 can each contact the patient's skin at or near the measurement site. The wider second portion 614 and the narrow first portion 612 of the sensor tape 600 can form independent, staggered loops around the patient's anatomy. In addition, the total contact area between the sensor tape 600 of the sensor assembly 70, 75 and the patient's skin is higher than the contact area between the sensor tape 200 and the patient's skin. The sensor assembly 70, 75 can therefore better secure the sensor 100 to the patient's skin than the sensor tape 200 and without requiring a significant increase in use of tape materials due to the first portion 612 of the sensor tape 600 being narrower and/or shorter than the second portion 614. Further, the first portion 612 of the sensor assembly 70 can attach a portion of the connector arm 120 to the patient's skin.

The staggered first and second portion 612, 614 can each form at least a first loop around the patient's anatomy without layer(s) of tape between the first or second portions 612, 614 and the patient's skin. The staggered first and second portion 612, 614 can thus result in even tape surfaces around the patient's anatomy. An even tape surface can provide better securement of the tape to the skin because there is no gap that could sometimes form when the adhesive side of a tape is placed partially over the skin and partially over a non-adhesive side of the previous loop of tape. The staggered first and second portion 612, 614 can also provide a mechanical decoupling along a joint of an appendage, such as a finger. The first and second portions 612, 614 can be placed above and below a joint respectively. The first and second portions 612, 614 can stay securely connected to the patient skin despite small movements of the patient, such as flexing of a finger or a foot, because the first and second portions 612, 614 are not connected along an entire length of the staggered sections. This can allow the finger to bend freely, but still maintain the tape in substantially the same position due to the increased adhesive surface provided by the first potion 612. As show in FIG. 14B, the detector arm 110 of the sensor 110 is placed beneath or immediately next to the adhesive side 606 of the sensor tape 600. The emitter 124 and the detector 113 can be aligned to the indicators 616, 618 respectively as discussed above to ensure that the detector arm 110 is placed within the boundary of the sensor tape 600. Placing the sensor 100 next to the adhesive side 606 of the sensor tape 600 can allow the sensor 100 and the tape 600 be assembled right before use. The sensor tape 600 can come in a variety of sizes, such as small, medium, and large. The appropriately sized sensor tape 600 can be selected depending on the size of the patient's anatomy. The separability of the sensor tape 600 from the sensor 100 can allow the sensor tape 600 be disposable so that a new sensor tape with a fresh adhesive side can be used for every measurement site to improve securement of the sensor to the measurement site. The separability of the sensor tape 600 from the sensor 100 can allow the more expensive components, such as the emitter 124, the detector 113, and other electrical components be reusable. Reusing the more expensive components can reduce cost of replacing the optical sensors.

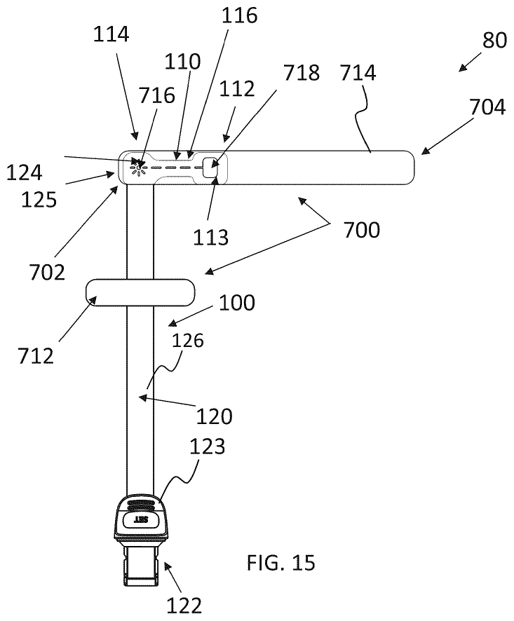

FIG. 15 illustrates a top view of a sensor assembly 80 including the L-shaped sensor 100 and a staggered sensor tape 700. The sensor tape 700 can have features of the sensor tapes 200, 300, 400, 500, 600 except as described below. Accordingly, features of the sensor tape 700 can be incorporated into features of the sensor tapes 200, 300, 400, 500, 600 and features of the sensor tapes 200, 300, 400, 500, 600 can be incorporated into features of the sensor tape 700. The sensor tape 700 can have a first portion 712 and a second portion 714. The first and second portions 712, 714 of the sensor tape 700 can each have an adhesive side and a non-adhesive side. The non-adhesive side of the second portion can include alignment indicators 716, 718. The second portion 714 can have a first end 702 and a second end 704 opposite the first end 702 along a length of the second portion 714. The first end 702 of the second portion 714 can be proximate the emitter 124. The indicator 716 can be aligned with the emitter 124. The sensor tape 700 can extend along the length of the detector arm 110, past the detector 113, and terminate at the second end 604. The sensor tape 700 can be substantially parallel to the detector arm 110. The indicator 718 can be aligned with the detector 113 of the L-shaped sensor 100. The alignment indicators 716, 718 can have the advantages described above. The first portion 712 can be detached from the second portion 714. The first portion 712 can be placed closer to the cable connector 123 than the second portion 714. The first portion 712 can be generally centered at the connector arm 120. A mid-point of the first portion 712 along a length of the first portion 712 can be offset from a midline along a length of the connector arm 120. The offset can be on the same side of the connector arm 120 as the detector 110 or on the opposite side. The first portion 712 can stabilize a portion of the connector arm 120 to the patient's skin, thereby facilitating the secure attachment of the sensor assembly 80 with the measurement site.

### Manufacturing of Sensor Tapes

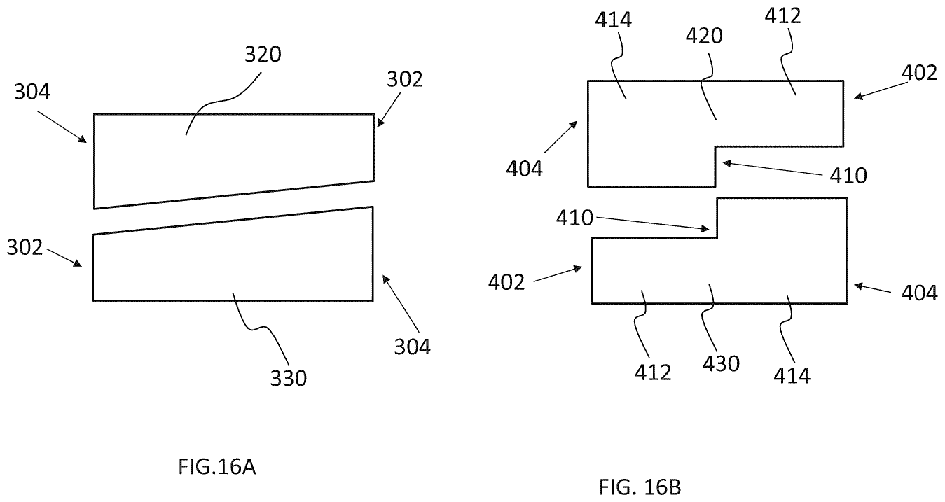

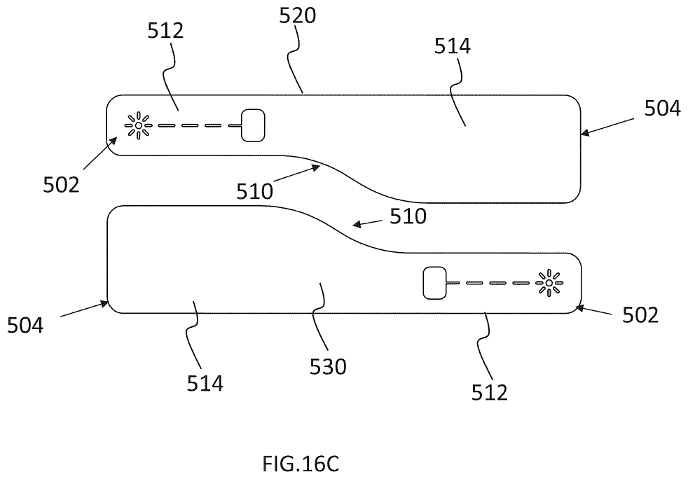

Certain manufacturing techniques for saving materials will now be described with reference to FIGS. 16A-C. As shown in FIG. 16A, during manufacturing, a top sensor tape 320 and a bottom sensor tape 330 can be cut from a rectangular piece of sensor tape material by a diagonal cut. The first and second ends of the bottom sensor tape 330 can be flipped horizontally in the cut pattern so that the first end 302 of the top sensor tape 320 aligns with the second end 304 of the bottom sensor tape 330 and the second end 304 of the top sensor tape 320 aligns with the first end 302 of the bottom sensor tape 330. As shown in FIG. 16B, during manufacturing, two pieces of the sensor tape 420, 430, each with the first and second portions 412, 414, can be cut from a rectangular piece of sensor tape material by a zig-zag lined cut. This manufacturing technique is especially advantageous if the first and second portions 412, 414 have the same length. The first and second ends of the bottom sensor tape 430 can be flipped in the cut pattern so that the first portion 412 of the top sensor tape 420 aligns with the second portion 414 of the bottom sensor tape 430 and the second portion 414 of the top sensor tape 420 aligns with the first portion 412 of the bottom sensor tape 430. Likewise, as shown in FIG. 16C, during manufacturing, two pieces of the sensor tape 520, 530 can be cut from a rectangular piece of sensor tape material by a zig-zag lined cut, especially if the first and second portions 512, 514 have the same length. The first and second ends of the bottom sensor tape 530 can be flipped in the cut pattern so that the first portion 512 of the top sensor tape 520 aligns with the second portion 514 of the bottom sensor tape 530 and the second portion 514 of the top sensor tape 520 aligns with the first portion 512 of the bottom sensor tape 530. As shown, the sensor tapes 300, 400, 500 can be manufactured with less waste in tape material despite the non-uniform widths of the tapes.

In some embodiments, the sensor assembly 10, 20, 25, 30, 35, 40, 45, 50, 55, 60, 65, 70, 75, 80 can have two layers of sensor tapes instead of only one layer of sensor tape 200, 300, 400, 500, 600, 700. The detector arm 100 can be sandwiched between the two layers of sensor tapes. The tape layer interfacing the detector arm 100 and the patient's skin can have two adhesive sides. The two layers of sensor tapes can have the same or different shapes and/or sizes.

In some embodiments, the sensor assembly 10, 20, 25, 30, 35, 40, 45, 50, 55, 60, 65, 70, 75, 80 can optionally include a liner and applicator tape (not shown). The liner can be printed with a variety of designs and/or colors. The liner can be long and wide enough to fit the length of the sensor tape 200, 300, 400, 500, 600, 700. The applicator tape can have a variety of shapes and sizes. In one embodiment, the applicator tape has a length and width that can fit onto the liner. Additional details regarding the liner and applicator tape and other features can be found in U.S. application Ser. No. 15/017,505, reference herein.

In some embodiments, the sensor tape 200, 300, 400, 500, 600, 700 can be used to secure any types of sensor to a patient's skin to form a sensor assembly. In some embodiments, the sensor tape 200, 300, 400, 500, 600, 700 can be used to secure any types of sensor to a surface of a medium other than a patient's skin to taking non-invasive measurement of characteristics of a medium.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. Additionally, as used herein, “gradually” has its ordinary meaning (e.g., differs from a non-continuous, such as a step-like, change).

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A noninvasive L-shaped sensor configured to be applied to a skin surface of a neonatal or infant patient for measuring physiological parameters of the neonatal or infant patient, the noninvasive L-shaped sensor comprising:
a flat detector strip comprising an optical emitter and an optical detector spaced along a longitudinal axis of the detector strip, the detector strip having a length along the longitudinal axis between a first end and a second end of the detector strip, wherein the detector strip is configured to be wrapped onto a body part of the neonatal or infant patient; and
a flat connector strip perpendicular to the longitudinal axis of the detector strip such that the connector strip extends away from the detector strip, a first end of the connector strip lengthwise being connected to the detector strip, the first end of the connector strip being aligned with the optical emitter, a second end of the connector strip opposite the first end of the connector strip terminating at a cable connector, an electrical circuit extending between the first and second ends of the connector strip, the electrical circuit electrically connecting the optical emitter, the optical detector, and the cable connector, wherein the cable connector is configured to be plugged into a corresponding port of a sensor cable, and wherein the connector strip comprises a flexible flat foam configured to protect the electrical circuit.

2. The noninvasive L-shaped sensor of claim 1, wherein the optical emitter is closer to the first end of the flat detector strip than the optical detector.

3. The noninvasive L-shaped sensor of claim 2, wherein, when in use, an initial portion of the flat detector strip starting from the first end of the detector strip and encompassing the optical emitter and the optical detector is configured to directly contact the skin surface.

4. The noninvasive L-shaped sensor of claim 3, wherein a terminal portion of the flat detector strip ending at the second end of the detector strip is configured to contact the initial portion.

5. The noninvasive L-shaped sensor of claim 1, wherein the length of the flat detector strip is at least twice of a distance between the optical emitter and the optical detector so as to wrap around the body part in more than one loop.

6. The noninvasive L-shaped sensor of claim 5, wherein the distance between the optical emitter and the optical detector is such that, when wrapped onto the body part of the neonatal or infant patient, the optical emitter and the optical detector are on opposite sides of the body part.

7. The noninvasive L-shaped sensor of claim 1, wherein the flat detector strip comprises a first surface and a second surface opposite the first surface, the first surface configured to face the skin surface and the second surface configured to face away from the skin surface.

8. The noninvasive L-shaped sensor of claim 7, wherein the second surface of the flat detector strip comprises a first alignment indicator aligned with the optical emitter and a second alignment indicator aligned with the optical detector.

9. The noninvasive L-shaped sensor of claim 7, wherein the first surface of the flat detector strip is an adhesive surface.

10. The noninvasive L-shaped sensor of claim 1, wherein the sensor is configured to be removably attached to the body part with hook and loop.

11. The noninvasive L-shaped sensor of claim 1, wherein the flat connector strip is rectangular in shape.

12. The noninvasive L-shaped sensor of claim 1, wherein the flat detector strip comprises a first edge and a second edge between the first and second ends of the detector strip, the second edge being opposite the first edge across a width of the detector strip, at least one of the first edge or the second edge being linear between the first end and the second end of the detector strip.

13. The noninvasive L-shaped sensor of claim 12, wherein the first edge and the second edge are linear between the first end and the second end of the detector strip such that the detector strip is rectangular in shape.

14. The noninvasive L-shaped sensor of claim 13, wherein another one of the first edge or the second edge is non-linear and includes a transition portion.

15. The noninvasive L-shaped sensor of claim 14, wherein the transition portion is a sloped transition portion.

16. The noninvasive L-shaped sensor of claim 1, wherein the optical emitter comprises more than one LED.
